(19) United States
(12) Patent Application Publication
(10) Pub. No.: US 2025/0381374 A1
(43) Pub. Date: Dec. 18, 2025

(54) OPTIMIZED BAV INFLATION DEVICE

(71) Applicant: BOSTON SCIENTIFIC SCIMED, INC., Maple Grove, MN (US)

(72) Inventors: Tim O'Connor, Galway (IE); Chris Cullen, Galway (IE); Gavin O'Brien, Dublin (IE); John Lardner, Galway (IE); Enda Hannon, Galway (IE)

(73) Assignee: BOSTON SCIENTIFIC SCIMED, INC., Maple Grove, MN (US)

(21) Appl. No.: 19/239,100

(22) Filed: Jun. 16, 2025

Related U.S. Application Data
(60) Provisional application No. 63/660,739, filed on Jun. 17, 2024.

(51) Int. Cl. A61M 25/10; A61M 25/00

(52) U.S. Cl. CPC A61M 25/10185; A61M 25/0043; A61M 2025/0001

(57) ABSTRACT

A balloon catheter inflation device includes at least a first barrel, a first piston, and a second piston. The first barrel has a first end coupled to an outlet chamber with a nozzle, and a second open end. The first piston is slidable within the first barrel and has a first end configured to frictionally engage an inner surface of the first barrel. The first piston is configured to apply a first pressure to fluid within the first barrel and the outlet chamber. The second piston is configured to apply a second pressure to the outlet chamber, where the second pressure is higher than the first pressure.

### CROSS-REFERENCE TO RELATED APPLICATION

This application is a continuation of U.S. Patent Application Ser. No. 63/660,739, filed Jun. 17, 2024, entitled “OPTIMIZED BAV INFLATION DEVICE”, which is incorporated by reference herein in its entirety.

### TECHNICAL FIELD

The disclosure pertains to medical devices and more particularly to balloon aortic valvuloplasty (BAV) devices utilized in the transcatheter implantation of prosthetic stent-valves, and methods for using such medical devices.

### BACKGROUND

A wide variety of medical devices have been developed for medical use including, for example, medical devices involved in transcatheter aortic valve replacement (TAVR). Balloon catheters may be utilized to pre-dilate the aortic valve before implanting a prosthetic stent-valve. In some cases, a balloon may also be used post implant to ensure optimal expansion and anchoring of the prosthetic stent-valve. Of the known medical devices and methods, each has certain advantages and disadvantages. There is an ongoing need to provide alternative medical devices as well as alternative methods for manufacturing and using the medical devices.

### SUMMARY

This disclosure provides design, material, manufacturing method, and use alternatives for medical devices. An example balloon catheter inflation device includes at least a first barrel having a first end coupled to an outlet chamber with a nozzle, and a second open end, a first piston slidable within the first barrel, the first piston having a first end configured to frictionally engage an inner surface of the first barrel, the first piston configured to apply a first pressure to fluid within the first barrel and the outlet chamber, and a second piston configured to apply a second pressure to the outlet chamber, wherein the second pressure is higher than the first pressure.

Alternatively, or additionally to the embodiment above, the balloon catheter inflation device further includes a check valve on the outlet chamber configured to limit a pressure within the outlet chamber.

Alternatively, or additionally to any of the embodiments above, the balloon catheter inflation device further includes a pressure gauge disposed on the outlet chamber and configured to measure a pressure within the outlet chamber.

Alternatively, or additionally to any of the embodiments above, the balloon catheter inflation device further includes a second barrel adjacent the first barrel, the second barrel having a first end coupled to the outlet chamber and a second open end, wherein the second piston is slidable within the second barrel.

Alternatively, or additionally to any of the embodiments above, the second piston has a resilient first end configured to have a friction fit within an inner surface of the second barrel.

Alternatively, or additionally to any of the embodiments above, when both the first and second pistons are in a retracted position, a piston head on a second end of the first piston is positioned higher than a piston head on a second end of the second piston.

Alternatively, or additionally to any of the embodiments above, a diameter of the first barrel is larger than a diameter of the second barrel.

Alternatively, or additionally to any of the embodiments above, the second open end of each of the first and second barrels is fixed to a flange.

Alternatively, or additionally to any of the embodiments above, the balloon catheter inflation device further includes a ratchet mechanism fixed to the flange and configured to engage notches in the first piston, the ratchet mechanism configured to move between a first position allowing free movement of the first piston within the first barrel, and a second position holding the first piston in place.

Alternatively, or additionally to any of the embodiments above, the first barrel defines a 60 ml volume and the second barrel defines a 10 ml volume.

Alternatively, or additionally to any of the embodiments above, the second piston is slidably disposed within an inner lumen of the first piston, and the first end of the first piston defines an opening.

Alternatively, or additionally to any of the embodiments above, the balloon catheter inflation device further includes a spring disposed around the second piston, positioned between a second end of the first piston and a piston head on the second piston.

Alternatively, or additionally to any of the embodiments above, the second piston engages an inner wall of the first piston such that when the nozzle of the balloon catheter inflation device is coupled to a balloon and the first and second pistons are in a retracted position, depressing the second piston first causes the first piston to advance through the first barrel to deliver fluid into the balloon.

Alternatively, or additionally to any of the embodiments above, when a first pressure is achieved within the balloon, further depressing the second piston causes the spring to collapse and the second piston to advance through the first piston to provide additional pressure to the balloon.

Alternatively, or additionally to any of the embodiments above, the first pressure is 1 atmosphere.

Alternatively, or additionally to any of the embodiments above, a first end of the second piston is configured to extend through the opening in the first end of the first piston.

Another example balloon catheter inflation device includes at least a first barrel having a first end coupled to an outlet chamber with a nozzle, and a second open end, wherein the outlet chamber includes a check valve and a pressure gauge, a first piston slidable within the first barrel, the first piston having a resilient first end configured to frictionally engage an inner surface of the first barrel, the first piston configured to apply a first pressure to fluid within the outlet chamber, and a second piston configured to increase a pressure in the outlet chamber after the first piston provides the first pressure.

Alternatively, or additionally to the embodiment above, the balloon catheter inflation device further includes a second barrel fixed to the first barrel, the second barrel having a first end fluidly coupled to the outlet chamber and a second open end, wherein the second piston is slidable within the second barrel to increase the pressure in the outlet chamber while the first piston remains static.

Alternatively, or additionally to any of the embodiments above, the first piston includes a lumen extending therethrough ending at an opening at the first end, and the second piston is slidably disposed within the lumen, wherein a spring is disposed around the second piston, positioned between a second end of the first piston and a piston head on the second piston, the spring configured to collapse once the second piston is depressed and 1 atmosphere of pressure is achieved in the outlet chamber.

An example method of inflating a balloon fixed to a distal end of a catheter using a balloon catheter inflation device includes attaching a nozzle of the balloon catheter inflation device to the catheter, the balloon catheter inflation device including, at least a first barrel having a first end coupled to an outlet chamber with the nozzle, and a second open end, a first piston slidable within the first barrel, the first piston having a first end configured to frictionally engage an inner surface of the first barrel, the first piston configured to apply a first pressure to fluid within the first barrel and the outlet chamber, and a second piston configured to apply a second pressure to the outlet chamber, wherein the second pressure is higher than the first pressure. the method of inflating the balloon further includes advancing the first piston within the first barrel to inflate the balloon to a first pressure, and advancing the second piston to pressurize the balloon to a second pressure greater than the first pressure.

The above summary of some embodiments, aspects, and/or examples is not intended to describe each embodiment or every implementation of the present disclosure. The figures and the detailed description which follows more particularly exemplify these embodiments.

### BRIEF DESCRIPTION OF THE DRAWINGS

The disclosure may be more completely understood in consideration of the following detailed description of various embodiments in connection with the accompanying drawings, in which:

FIG. 1 is a graph showing the plunger force (y axis) required to achieve various pressures in a balloon using two sizes of syringes (x axis);

FIGS. 2A and 2B are partial cross-sectional views illustrating a first embodiment of a dual piston inflation device with a first piston in first and second positions, respectively;

FIG. 3 is a perspective view of a second embodiment of a dual piston inflation device;

FIGS. 4A and 4B are partial cross-sectional views illustrating details of the inflation device of FIG. 3 in first and second configurations;

FIG. 5 is a partial cross-sectional view illustrating an alternative inner plunger configuration for another inflation device;

FIG. 6A is a partial cross-sectional view illustrating another embodiment of a dual piston inflation device, with the piston head removed; and

FIG. 6B is a cross-sectional view taken along line 6B-6B in FIG. 6A.

While aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5). Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

Relative terms such as “proximal”, “distal”, “advance”, “withdraw”, variants thereof, and the like, may be generally considered with respect to the positioning, direction, and/or operation of various elements relative to a user/operator/manipulator of the device, wherein “proximal” and “withdraw” indicate or refer to closer to or toward the user and “distal” and “advance” indicate or refer to farther from or away from the user. In some instances, the terms “proximal” and “distal” may be arbitrarily assigned in an effort to facilitate understanding of the disclosure, and such instances will be readily apparent to the skilled artisan.

Additionally, the term “substantially” when used in reference to two dimensions being “substantially the same” shall generally refer to a difference of less than or equal to 5%. The terms “monolithic” and “unitary” shall generally refer to an element or elements made from or consisting of a single one-piece structure. A monolithic and/or unitary element shall exclude structure and/or features made by assembling or otherwise joining multiple discrete elements together.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to affect the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

The following description should be read with reference to the drawings, which are not necessarily to scale, wherein similar elements in different drawings are numbered the same. The detailed description and drawings are intended to illustrate but not limit the disclosure. Those skilled in the art will recognize that the various elements described and/or shown may be arranged in various combinations and configurations without departing from the scope of the disclosure. The detailed description and drawings illustrate example embodiments of the disclosure. However, in the interest of clarity and ease of understanding, while every feature and/or element may not be shown in each drawing, the feature(s) and/or element(s) may be understood to be present regardless, unless otherwise specified.

Pre-dilatation of the native valve using an expandable balloon is often recommended for transcatheter aortic valve replacement (TAVR) procedures when using a self-expanding TAVR valve, usually of a diameter one millimeter less than the perimeter of the native valve annulus, in order to prepare the anatomy for optimal valve implantation and to achieve a maximized post-implant effective orifice area. Pre-dilation may be performed using a balloon aortic valvuloplasty (BAV) balloon. Also, during some TAVR procedures, it may be necessary to post dilate the implanted valve again with a BAV device to optimize the expansion of the prosthesis. This may be required for a heavily calcified anatomy.

The majority of currently available BAV devices have a nominal diameter pressure rating of 4 atmospheres (atm) and a rated burst pressure of 6 atm, for example. Accurately reaching these pressures often involves the use of an inflation device with a gauge and threaded piston, as commonly used in coronary angioplasty and stenting. However, this inflation method takes significant time and consequently extends the length of time that the patient's annulus is blocked with the inflated balloon. This also requires extending the period of rapid pacing during the dilation step. This is not suitable for frail patients with low ejection fraction and advanced disease.

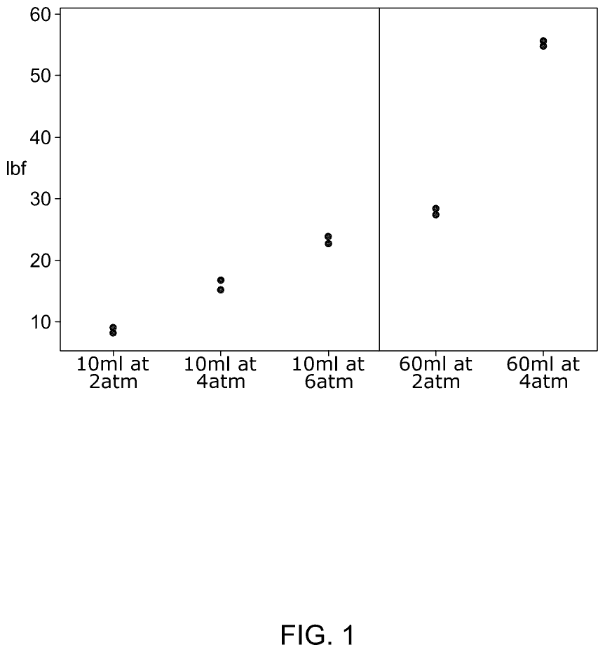

To minimize the inflation/deflation and rapid pacing time (to generally less than three seconds), many operators currently use a large volume syringe (50 milliliter (ml) to 60 ml) to inflate/deflate the BAV balloon. A significant problem with this technique is that it requires significant hand grip force to achieve the desired inflation pressure of between 4 atm and 6 atm (rated and burst pressure) with a large bore syringe (50 ml to 60 ml), which often results in the optimal pressure for pre and/or post dilation not being achieved. FIG. 1 is a graph showing the force required to achieve optimal inflation pressure in a balloon used for BAV. The graph plots the plunger force, in pound force (lbf) on the y axis, required to achieve 2, 4, and 6 atm of pressure on x axis, using a 10 ml or 60 ml syringe. The data from FIG. 1 is shown below in Table 1.

TABLE 1

| 10 ml at 2 | 10 ml at 4 | 10 ml at 6 | 60 ml at 2 | 60 ml at 4 |
|---|---|---|---|---|
| atm | atm | atm | atm | atm |
| 8.885 | 15.175 | 23.770 | 27.960 | 55.475 |
| 8.595 | 16.855 | 22.735 | 28.305 | 54.840 |
| 8.185 | 16.590 | 23.355 | 27.325 | 54.990 |

Three measurements were taken to achieve each pressure point. As seen in the left side of the graph, and as shown in Table 1, the 10 ml syringe can achieve 2 atm, 4 atm, and 6 atm comfortably by hand, at about 8 lbf, 16 lbf, and 23 lbf respectively. However, it is much more difficult to achieve the desired pressure with a 60 ml syringe. As seen in the right side of the graph, the force required to get to 2 atm with a 60 ml syringe is about 28 lbf and to achieve 4 atm it takes about 55 lbf, which is very difficult by hand. It is practically impossible to achieve 6 atm by hand with a 60 ml syringe.

In order to achieve the desired balloon pressure, some operators use a smaller conventional syringe connected in parallel with a larger conventional syringe but this requires additional steps and components like stop cocks connected to switch between syringes. While such systems may require less hand grip force to achieve the desired inflation pressure, the procedure also takes longer, thus extending the time the annulus is blocked. Significantly less hand grip force is required to achieve between 4 atm and 6 atm with a smaller bore syringe, for example a 10 ml syringe, however this volume is often too small to fill and then pressurize the BAV balloon, which may have a volume of from 20 ml to 80 ml for a large bore balloon and from 5 ml to 20 ml for a small bore balloon.

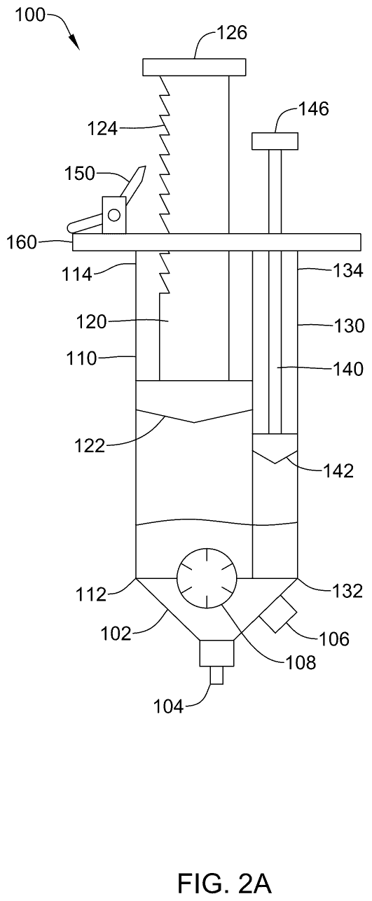

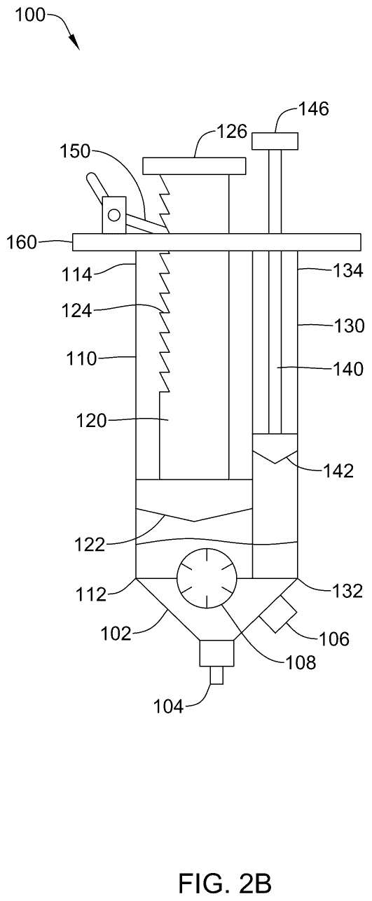

A combination of two syringe mechanisms has been developed which provides for both the fill and pressure targeting features in one device that is achieved with lower hand grip force. FIG. 2A illustrates one embodiment of a balloon catheter inflation device 100 including a first barrel 110 having a first end 112 coupled to an outlet chamber 102 with a nozzle 104, and an open second end 114. A first piston 120 may be slidable within the first barrel 110, and the first piston 120 may have a first end 122 configured to frictionally engage an inner surface of the first barrel to form a fluid-tight seal. The first end 122 may be made of a compressible and resilient material such as an elastomer. The first piston 120 may be configured to apply a first pressure to fluid within the first barrel 110 and the outlet chamber 102. The device may further include a second barrel 130 parallel to and fixed to the first barrel 110. The first and second barrels 110, 130 may define separate volumes, but are both in fluid communication with the outlet chamber 102 and nozzle 104. The second barrel 130 has a first end 132 coupled to the outlet chamber 102 and an open second end 134. A second piston 140 may be slidable within the second barrel 130. The second piston may have a resilient first end 142 configured to have a friction fit within an inner surface of the second barrel. The first barrel 110 may have a larger diameter than the second barrel 130, resulting in the second piston 140 being configured to apply a second pressure to the outlet chamber 102 that is higher than the first pressure applied by the first piston 120 in the first barrel. The first barrel 110 may have an inner diameter of 25 mm to 30 mm and the second barrel 130 may have an inner diameter of 14 mm to 16 mm. In some embodiments, the first barrel 110 may have a volume of 50 ml or 60 ml and the second barrel 130 may have a volume of 10 ml. The larger first barrel 110 and piston may be used to inflate the balloon and the smaller second barrel 130 and piston may be used to apply and regulate a higher pressure needed to pressurize the balloon to the desired pre-dilation or post-dilation pressure. In some embodiments, the second barrel 130 and second piston 140 may be used to provide a desired pressure in the balloon of 4 atm. The outlet chamber 102 may include a check valve 106 configured to limit the pressure within the outlet chamber. For example, the check valve 106 may open at or below the rated burst pressure, for example at 6 atm, to allow the inflation media to leak out through the check valve so that the outlet chamber pressure can never go above the rated burst pressure of the balloon. In some embodiments, a pressure gauge 108 may be disposed on the outlet chamber 102 and configured to measure a pressure within the outlet chamber 102.

Before inflating the balloon, both the first and second pistons 120, 140 are in a retracted position, and the piston head 126 on the second end of the first piston 120 may be positioned higher than the piston head 146 on the second end of the second piston 140, as shown in FIG. 2A. In this position, the user easily knows to depress the first piston 120 first to inflate the balloon, and after the balloon is inflated to the desired pressure, the second piston 140 is depressed to pressurize the balloon.

The open second end 114 of the first barrel 110 and the open second end 134 of the second barrel 130 may be fixed to a flange 160 extending radially outward from the barrels. A ratchet mechanism 150 may be fixed to the flange adjacent the first piston 120, which may have a series of notches 124 on at least the region of the first piston 120 adjacent the ratchet mechanism. The ratchet mechanism 150 may include a lever configured to engage the notches 124. The ratchet mechanism 150 may be configured to move between a first position allowing free axial movement of the first piston 120 within the first barrel, and a second position holding the first piston in place.

Once the first piston 120 has been advanced within the first barrel 110 to inflate the balloon, the ratchet mechanism 150 may engage one of the notches 124, as shown in FIG. 2B. The ratchet mechanism may hold the first piston so further pressure on the piston head 126 cannot move the first piston further into the first barrel 110. Additionally, the ratchet mechanism may also hold the first piston 120 in a fixed position such that when the piston head 146 of the second piston 140 is depressed, the increased pressure in the outlet chamber 102 cannot cause the first piston 120 to be pushed upwards and out of the first barrel 110. The ratchet mechanism 150 may be manually disengaged in order to quickly retract the first piston 120 to rapidly deflate the balloon.

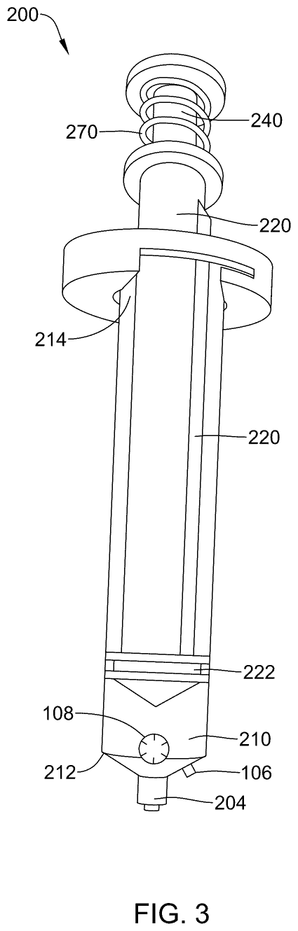

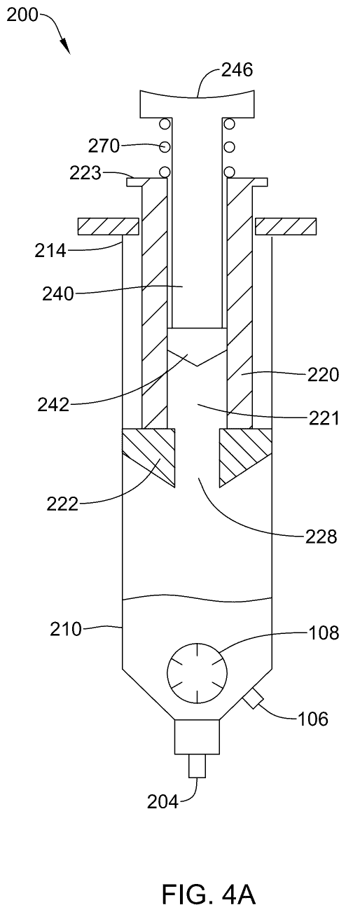

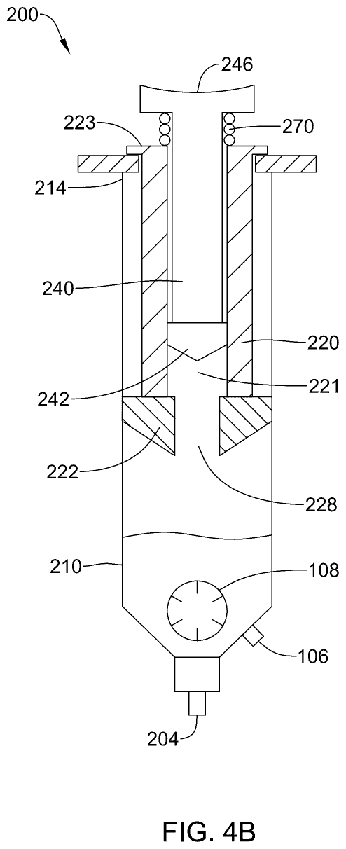

FIGS. 3, 4A, and 4B illustrate another embodiment of balloon catheter inflation device 200. Similar to the above described embodiment, the device 200 includes only a first barrel 210 having a first end 212 with a nozzle 204, check valve 106, and optional pressure gauge 108. The second end 214 is open to receive the first piston 220, which may be slidable within the first barrel 210. The first piston may have a first end 222 configured to frictionally engage an inner surface of the first barrel. The first end 222 may be made of a resilient material. The first piston 220 may be configured to apply a first pressure to fluid within the first barrel 210 and exiting the nozzle 204. In this embodiment, a second piston 240 with a resilient first end 242 is slidably disposed within a lumen 221 extending through the first piston 220 and defining an opening 228 through the first end 222 of the first piston.

With this nested piston configuration, the larger first piston 220 may be advanced towards the nozzle 204 to provide the initial, lower pressure needed to inflate the balloon, and after inflation, the smaller second piston 240 may be advanced towards the nozzle 204 to apply and regulate the higher pressure needed to pressurize the balloon. This is achieved via a spring 270 disposed around the second piston 240 and positioned between a second end 223 of the first piston 220 and the piston head 246 on the second piston 240. The first end 242 of the second piston 240 is configured to engage the inner wall of the lumen 221 through the first piston in a fluid-tight seal. When the first and second pistons 220, 240 are in a retracted position, the spring 270 is in a relaxed, expanded configuration, as shown in FIG. 4A. When the piston head 246 of the second piston is initially depressed, the spring 270 prevents the second piston 240 from advancing into the first piston 220, and instead causes the first piston 220 to advance through the first barrel 210 to deliver fluid through the nozzle 204 into a coupled balloon. When a desired first pressure is achieved within the balloon, additional force applied to the piston head 246 causes the spring 270 to collapse which causes the second piston to advance through the first piston to provide additional pressure to the balloon, while the first piston remains static and does not advance, as shown in FIG. 4B. The desired first pressure at which the spring collapses may be between 0.5 atm and 2 atm. In some embodiments the desired first pressure may be 1 atm. The spring 270 prevents the second piston 240 from being advanced through the first piston 220 until the desired pressure in the balloon is reached. In this embodiment, the second piston 240 only exerts pressure on fluid within the first barrel 210 once the pressure in the first barrel 210 is greater than 1 atm. The second piston 240 may be advanced until the desired pressure in the balloon is achieved, such as 4 atm. In an alternative embodiment, instead of the spring 270, a fluid orifice may be used like a damper (not shown).

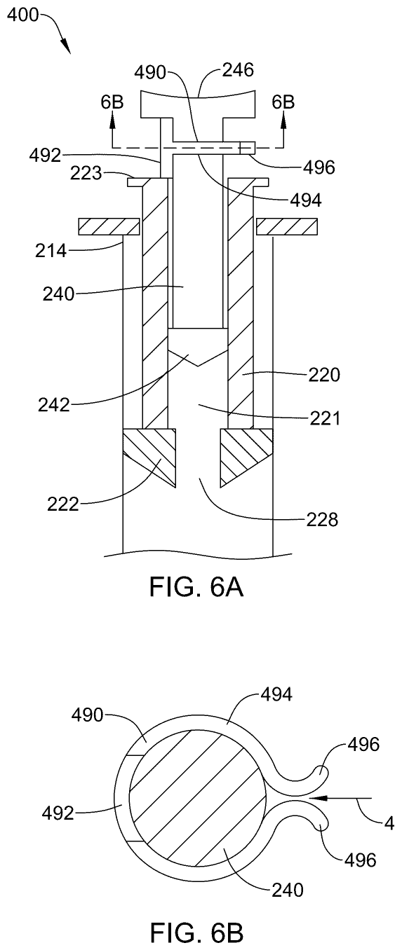

In a further embodiment, instead of the spring 270, a clip 490 may be used to prevent the second piston 240 from being depressed while the first piston 220 is depressed to inflate the balloon. FIG. 6A shows an embodiment having the same structure as in FIG. 4A with the only change being the spring 270 has been replaced with a clip 490 disposed around the upper end of the second piston 240. FIG. 6B is a cross-sectional view taken along line 6B-6B in FIG. 6A, with the piston head 246 removed for clarity. The clip 490 includes a substantially circular main body 494 that surrounds the second piston 240, as shown in FIG. 6B. The main body 494 has two free ends 496 that are curved away from one another and extend laterally outward from the substantially circular portion of the main body 494. The clip 490 includes a vertical bar 492 extending perpendicular to the main body 494. The vertical bar 492 is configured to hold the piston head 246 spaced apart from the second end 223 of the first piston 220, and prevent any distal advancement of the second piston 240 into the first piston 220 when the piston head 246 is depressed. See FIG. 6A. The main body 494 may be made of a material that allows for manual separation of the curved free ends 496 to allow the clip 490 to be removed from the second piston 240. The main body 494 is biased in the closed position as shown in FIG. 6B, with a snug fit around the second piston 240. Once the balloon has been inflated by depressing the piston head 246 and thereby depressing the first piston 220, the clip 490 may be removed by pressing against the inside edges of the free ends 496, as indicated by arrow 4, thereby separating the free ends 496 enough to allow the user to manually remove the clip 490. Once the clip 490 has been removed, further depression of the piston head 246 moves the second piston 240 into the first piston 220, thereby increasing the pressure in the balloon. After the balloon has been pressurized, the clip 490 may be reattached to the second piston 240 if the device is to be reused.

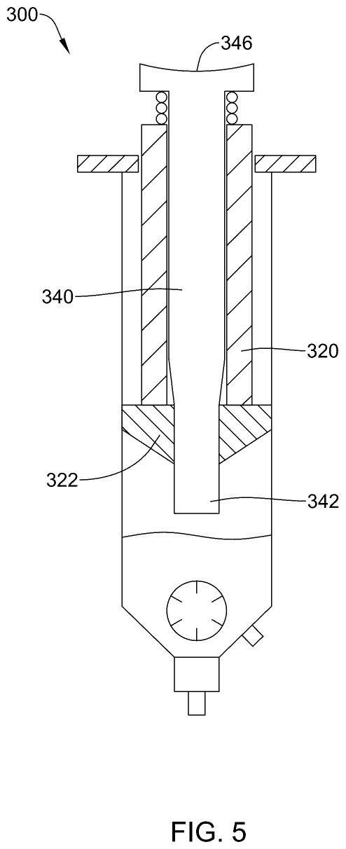

In another embodiment of balloon catheter inflation device 300, similar to the device 200, the inner pressure regulating component may be a second plunger 340 instead of a piston as described above. The the first end 342 of the second plunger 340 may be configured to extend completely through the opening in the first end 322 of the first piston 320 when the second plunger 340 is advanced to pressurize the balloon. See FIG. 5. This device 300 may be similar to or the same as the device 200 in all other structure and mode of operation.

A method of inflating a balloon fixed to a distal end of a catheter may be performed using any of the above described balloon catheter inflation devices. The method may involve attaching the nozzle of the balloon catheter inflation device to the catheter, advancing the first piston within the first barrel to inflate the balloon to a first pressure, and advancing the second piston to pressurize the balloon to a second pressure greater than the first pressure. When using the inflation device 100 illustrated in FIGS. 2A and 2B, advancing the first piston 120 includes depressing the piston head 126 on the first piston 120, and advancing the second piston 140 includes depressing the piston head 146 on the second piston 140. When using the inflation devices 200 and 300 illustrated in FIGS. 4A-5, advancing the first piston 220, 320 includes depressing the piston head 246, 346 on the second piston 240/second plunger 340, and advancing the second piston includes further depressing the piston head 246, 346 on the second piston 240/second plunger 340 until the spring 270 collapses and allows the second piston 240/second plunger 340 to advance within the lumen of the first piston 220, 320.

It will be understood that the pressures described in association with the above figures are illustrative only, and that other pressures are contemplated. The materials that can be used for the various components of the balloon catheter inflation device 100, 200, 300, and the various elements thereof disclosed herein may include those commonly associated with medical devices. For simplicity purposes, the following discussion refers to the inflation device 100 (and variations, systems or components disclosed herein). However, this is not intended to limit the devices and methods described herein, as the discussion may be applied to other elements, members, components, or devices disclosed herein.

In some embodiments, the resilient elements, including the piston first ends 122, 142, 222, 242, 322, may be made of compressible, elastomeric materials. Some examples of suitable compressible, elastomeric materials include a thermoset rubber (e.g., butyl rubber), a thermoplastic elastomer (TPE), thermoplastic polyurethane (TPU), silicone, and silicone rubber. In some examples the elastomeric material may have a durometer of about 20 to about 90 when measured on the Shore A scale. The remaining elements of the balloon catheter inflation device 100 (and variations, systems or components thereof disclosed herein) may be made from a polymer or other suitable material generally used for medical catheters. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly (alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex® high-density polyethylene, Marlex® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro (propyl vinyl ether) (PFA), ethylene vinyl alcohol, polyolefin, polystyrene, epoxy, polyvinylidene chloride (PVdC), poly (styrene-b-isobutylene-b-styrene) (for example, SIBS and/or SIBS 50A), polycarbonates, ionomers, polyurethane silicone copolymers (for example, Elast-Eon® from AorTech Biomaterials or ChronoSil® from AdvanSource Biomaterials), biocompatible polymers, other suitable materials, or mixtures, combinations, copolymers thereof, polymer/metal composites, and the like. In some embodiments, the sheath can be blended with a liquid crystal polymer (LCP). For example, the mixture can contain up to about 6 percent LCP.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The disclosure's scope is, of course, defined in the language in which the appended claims are expressed.

What is claimed is:

1. A balloon catheter inflation device, comprising:
at least a first barrel having a first end coupled to an outlet chamber with a nozzle, and a second open end;
a first piston slidable within the first barrel, the first piston having a first end configured to frictionally engage an inner surface of the first barrel, the first piston configured to apply a first pressure to fluid within the first barrel and the outlet chamber; and
a second piston configured to apply a second pressure to the outlet chamber, wherein the second pressure is higher than the first pressure.

2. The balloon catheter inflation device of claim 1, further comprising a check valve on the outlet chamber configured to limit a pressure within the outlet chamber.

3. The balloon catheter inflation device of claim 1, further comprising a pressure gauge disposed on the outlet chamber and configured to measure a pressure within the outlet chamber.

4. The balloon catheter inflation device of claim 1, further comprising a second barrel adjacent the first barrel, the second barrel having a first end coupled to the outlet chamber and a second open end, wherein the second piston is slidable within the second barrel.

5. The balloon catheter inflation device of claim 4, wherein the second piston has a resilient first end configured to have a friction fit within an inner surface of the second barrel.

6. The balloon catheter inflation device of claim 4, wherein when both the first and second pistons are in a retracted position, a piston head on a second end of the first piston is positioned higher than a piston head on a second end of the second piston.

7. The balloon catheter inflation device of claim 4, wherein a diameter of the first barrel is larger than a diameter of the second barrel.

8. The balloon catheter inflation device of claim 4, wherein the second open end of each of the first and second barrels is fixed to a flange.

9. The balloon catheter inflation device of claim 8, further comprising a ratchet mechanism fixed to the flange and configured to engage notches in the first piston, the ratchet mechanism configured to move between a first position allowing free movement of the first piston within the first barrel, and a second position holding the first piston in place.

10. The balloon catheter inflation device of claim 4, wherein the first barrel defines a 60 ml volume and the second barrel defines a 10 ml volume.

11. The balloon catheter inflation device of claim 1, wherein the second piston is slidably disposed within an inner lumen of the first piston, and the first end of the first piston defines an opening.

12. The balloon catheter inflation device of claim 11, further comprising a spring disposed around the second piston, positioned between a second end of the first piston and a piston head on the second piston.

13. The balloon catheter inflation device of claim 12, wherein the second piston engages an inner wall of the first piston such that when the nozzle of the balloon catheter inflation device is coupled to a balloon and the first and second pistons are in a retracted position, depressing the second piston first causes the first piston to advance through the first barrel to deliver fluid into the balloon.

14. The balloon catheter inflation device of claim 13, wherein when a first pressure is achieved within the balloon, further depressing the second piston causes the spring to collapse and the second piston to advance through the first piston to provide additional pressure to the balloon.

15. The balloon catheter inflation device of claim 14, wherein the first pressure is 1 atmosphere.

16. The balloon catheter inflation device of claim 14, wherein a first end of the second piston is configured to extend through the opening in the first end of the first piston.

17. A balloon catheter inflation device, comprising:
at least a first barrel having a first end coupled to an outlet chamber with a nozzle, and a second open end, wherein the outlet chamber includes a check valve and a pressure gauge;
a first piston slidable within the first barrel, the first piston having a resilient first end configured to frictionally engage an inner surface of the first barrel, the first piston configured to apply a first pressure to fluid within the outlet chamber; and
a second piston configured to increase a pressure in the outlet chamber after the first piston provides the first pressure.

18. The balloon catheter inflation device of claim 17, further comprising a second barrel fixed to the first barrel, the second barrel having a first end fluidly coupled to the outlet chamber and a second open end, wherein the second piston is slidable within the second barrel to increase the pressure in the outlet chamber while the first piston remains static.

19. The balloon catheter inflation device of claim 17, wherein the first piston includes a lumen extending therethrough ending at an opening at the first end, and the second piston is slidably disposed within the lumen, wherein a spring is disposed around the second piston, positioned between a second end of the first piston and a piston head on the second piston, the spring configured to collapse once the second piston is depressed and 1 atmosphere of pressure is achieved in the outlet chamber.

20. A method of inflating a balloon fixed to a distal end of a catheter using a balloon catheter inflation device, comprising:
attaching a nozzle of the balloon catheter inflation device to the catheter, the balloon catheter inflation device including:
at least a first barrel having a first end coupled to an outlet chamber with the nozzle, and a second open end;
a first piston slidable within the first barrel, the first piston having a first end configured to frictionally engage an inner surface of the first barrel, the first piston configured to apply a first pressure to fluid within the first barrel and the outlet chamber; and
a second piston configured to apply a second pressure to the outlet chamber, wherein the second pressure is higher than the first pressure;
advancing the first piston within the first barrel to inflate the balloon to a first pressure; and
advancing the second piston to pressurize the balloon to a second pressure greater than the first pressure.
